(19) United States
(12) Patent Application Publication
(10) Pub. No.: US 2014/0120607 A1
(43) Pub. Date: May 1, 2014

(54) Multi-Layered Cell Culture Vessel

(71) Applicant: COMING INCORPORATED, Coming, NY (US)

(72) Inventor: Elizabeth J. Abraham, Andover, MA (US)

(73) Assignee: CORNING INCORPORATED, Corning, NY (US)

(21) Appl. No.: 13/832,739

(22) Filed: Mar. 15, 2013

Related U.S. Application Data
(60) Provisional application No. 61/719,156, filed on Oct. 26, 2012.

(51) Int. Cl. C12M 1/00

(52) U.S. Cl. CPC C12M 23/34
USPC 435/289.1

(57) ABSTRACT

A multi-layered cell culture vessel is provided herein which includes a body enclosing a closed volume. The body includes at least one insert layer located within the closed volume to separate portions thereof, and at least one port allowing communication with the closed volume from exteriorly of the body. At least one grip may be provided which protrudes from the body. Additional features may include the use of a side port which is disposed transversely to a longitudinal axis of the body, including being disposed to intersect a gap located adjacent to each of the insert layers. This arrangement allows for the preparation and removal of cell culture medium without obstruction from the insert layers. Additionally, the end of the body may be formed with tapered portions that channel flow towards an end port thereby allowing for a relatively well-defined stream to be poured from the device as needed.

### CROSS-REFERENCE TO RELATED APPLICATION

This application claims priority to U.S. Provisional Patent Application No. 61/719,156, filed on Oct. 26, 2012, the entire contents of which are incorporated by reference herein.

### FIELD OF THE INVENTION

The application relates to multi-layered cell culture vessels and, more particularly, to closed-volume multi-layered cell culture vessels which are accessed through a port of limited diameter.

### BACKGROUND OF THE INVENTION

Multi-layered cell culture vessels are known in the prior art, including that disclosed in U.S. Published Patent Application No. 2011/0020923, to the Assignee herein. U.S. Published Patent Application No. 2011/0020923 is incorporated by reference herein. Multi-layered cell culture vessels provide a higher surface to volume ratio for a cell culture for a given footprint as compared to non-layered, standard open volume cell culture flasks and similar structures. Improvements in the ability to handle multi-layered cell culture vessels, as well as, in the ability to prepare and remove cultured cells in and from, respectively, multi-layered cell culture vessels are desirable.

### SUMMARY OF THE INVENTION

In one aspect, a multi-layered cell culture vessel is provided herein which includes a body enclosing a closed volume. The body includes at least one insert layer located within the closed volume to separate portions thereof, and at least one port allowing communication with the closed volume from exteriorly of the body. At least one grip protrudes from the body configured to be grippingly engaged by a user in transporting the multi-layered cell culture vessel. Advantageously, an external grip is provided herein which permits for better handling than with traditional multi-layered cell culture vessels.

Additional features are disclosed herein including the use of a side port which is disposed transversely to a longitudinal axis of the body, including being disposed to intersect a gap located adjacent to each of the insert layers. This arrangement allows for the preparation and removal of cell culture medium without obstruction from the insert layers. Additionally, the end of the body may be formed with tapered portions that channel flow towards an end port thereby allowing for a relatively well-defined stream to be poured from the device as needed.

These and other features of the invention will be better understood through a study of the following detailed description and accompanying drawings.

### BRIEF DESCRIPTION OF THE DRAWINGS

FIG. 1 is a perspective view of a multi-layered cell culture vessel formed in accordance with the subject invention;

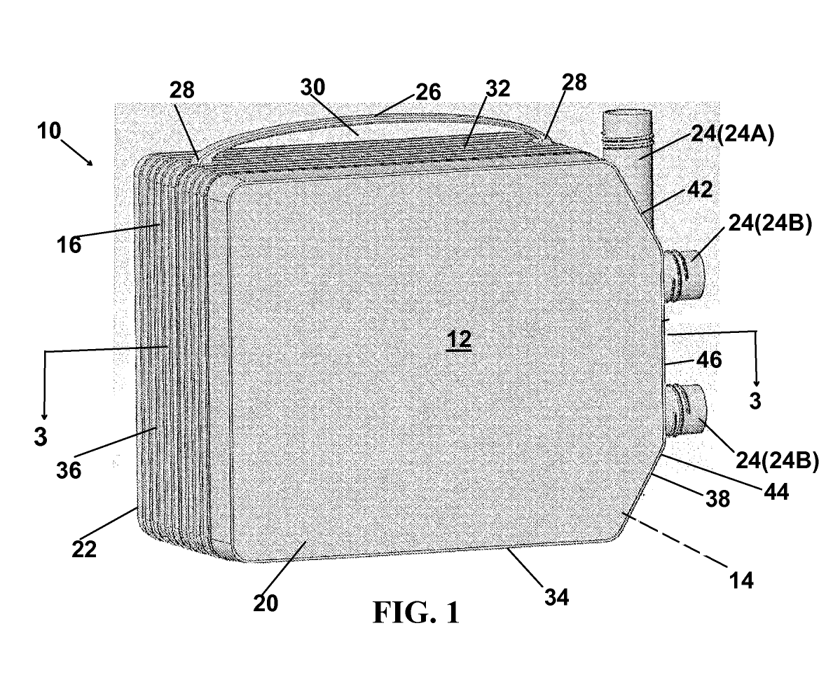

FIG. 2 is a side elevational view of a multi-layered cell culture vessel formed in accordance with the subject invention; and,

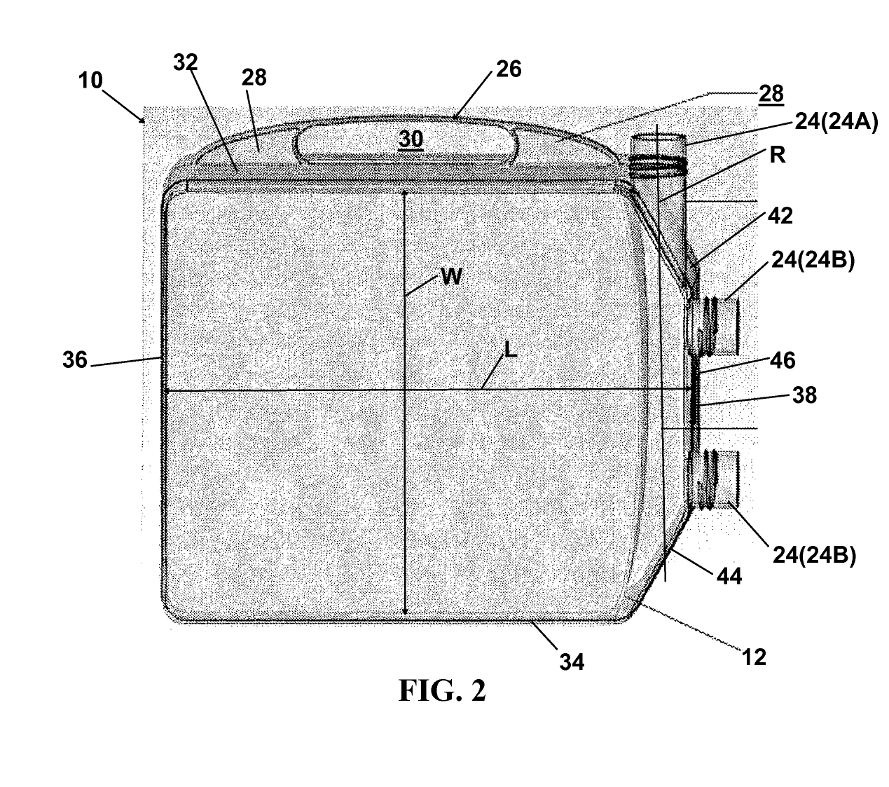

FIG. 3 is a cross-sectional view taken along line 3-3 of FIG. 1.

### DETAILED DESCRIPTION OF THE INVENTION

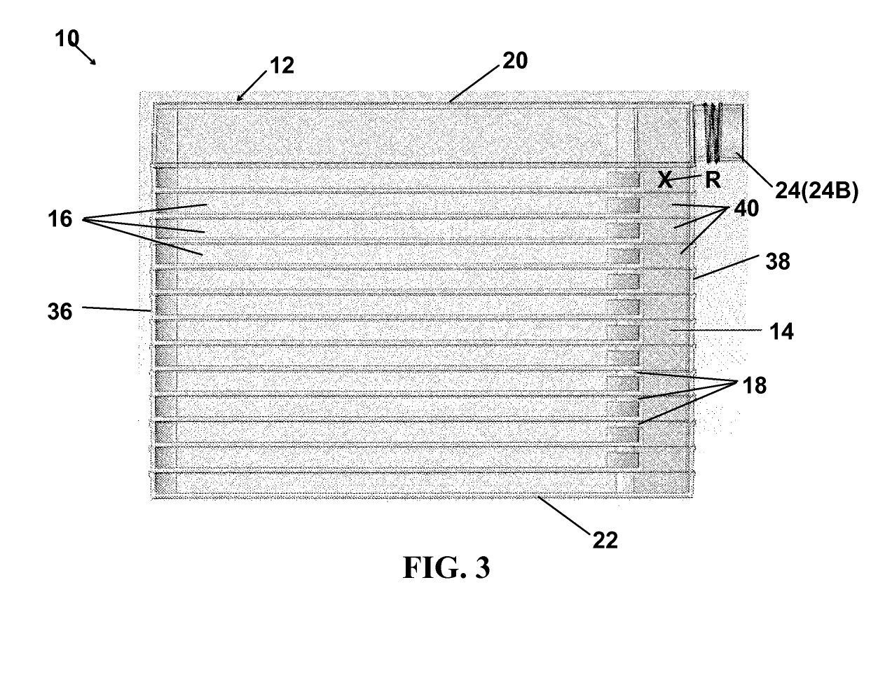

With reference to the Figures, a multi-layered cell culture vessel is shown and generally designated with the reference numeral 10. The vessel 10 includes a body 12 which encloses a closed volume 14. At least one insert layer 16 is located within the closed volume 14 to separate portions thereof. The insert layers 16 provide additional surface area within the closed volume 14 for cell culturing. As will be appreciated by those skilled in the art, the insert layers 16 may be of various configurations, and provided in various quantities, including being in a stacked configuration, as best shown in FIG. 3. Preferably, one or more openings 18 are provided between, or through, the insert layers 16 so as to allow for gas transmission throughout the closed volume 14, without obstruction of the insert layers 16. The body 12 may also include a top 20, a bottom 22, and side walls, as needed to enclose the closed volume 14. The side walls may be defined by portions of the insert layers 16, the top 20 and/or the bottom 22. Alternatively, separate side walls located about the insert layers 16, the top 20 and/or the bottom 22 may be utilized.

At least one port 24 is provided with the body 12 to allow communication with the closed volume 14 from exteriorly of the body 12. The port 24 may be in the form of one or more side ports 24A and/or in the form of one or more end ports 24B.

To facilitate handling of the vessel 10, at least one grip 26 may be provided which protrudes from the body 12 and is configured to be grippingly engaged by a user in transporting the vessel 10. As will be appreciated by those skilled in the art, the grip 26 may be formed with various configurations. In a preferred arrangement, the grip 26 is arcuately formed with ends 28 being fixed to the body 12 and a space 30 being defined between the grip 26 and the body 12 configured to receive a hand of a user.

The body 12 preferably defines a generally rectangular footprint as viewed in a first direction with a longitudinal dimension L which is preferably greater than a transverse dimension W. Accordingly, the body 12 includes first and second sides 32, 34 which are generally parallel to the longitudinal dimension L, and first and second ends 36, 38 which are generally parallel to the transverse dimension W. Preferably, the grip 26 is located on the first side 32 with at least one of the end ports 24B being located on the second end 38. This allows for the grip 26 to be used to conveniently pour cell media from the vessel 10 via the end port(s) 24B.

At least one of the side ports 24A may protrude from the body 12. The side port 24A may be generally tubular and extend along a secondary longitudinal axis R which is disposed generally parallel to the transverse dimension W. As known in the art, a cap, or other sealing member, may be mounted to the side port 24A to cause selective sealing thereof. The cap may be vented or solid to permit or prevent gas transmission therethrough, as needed. In addition, tubing or other instrumentation may be passed through the cap. The side port 24A may protrude from the body 12 in a direction generally parallel to the direction of the grip 26. It is preferred that the grip 26 protrude from the body 12 no further than the side port 24A with a cap mounted thereto.

The use of the one or more side ports 24A and/or one or more of the end ports 24B permits for gas exchange through multiple directions within the closed volume 14. The ports 24A, 24B may be in varying combinations provided with vented or closed caps to control gas transmission and flow.

In addition, the insert layers 16 may be formed to not extend fully across the body 12. With reference to FIG. 3, the insert layers 16 may extend from the first end 36 of the body and be spaced from the second end 38 so as to each define a gap 40 therebetween. The gaps 40 are preferably axially aligned. One or more of the gaps 40 is located to be intersected by the secondary longitudinal axis R. The gaps 40 collectively provide access across the closed volume 14, particularly in the transverse dimension W.

Also, the one or more side ports 24A may be used to prepare a cell culture within the vessel 10 with media and/or reagents being introduced into the closed volume 14, either by pouring, pipette or tubing, through the one or more side ports 24A. The filling can be accomplished either by gravity filling or pump-feeding. It is preferred to fill the vessel 10 with any media or reagents with the body 12 resting on the second side 34 so that the one or more side ports 24A protrude upwardly. This presents an equilibration position of the vessel 10 where introduced media and reagents are caused to collect in the closed volume 14 as a volume adjacent to the second side 34. In this position, the introduced media and reagents equilibrate across the insert layers 16. Once filled, the grip 26 may be used to transport the vessel 10 to a desired location, such as an incubator. Once at the desired location, and possibly using the grip 26, the body 12 is re-oriented to rest on the bottom 22. With re-orientation, the media partitions amongst the insert layers 16 in even amounts. Content may be removed through one or more of the end ports 24B by pouring or using tubing with gravity or pump extraction. The vessel 10 may be stood on an inclined support to assist in draining.

In a preferred arrangement, the second end 38 may be formed to channel flow towards the one or more end ports 24B. The second end 38 may include first and second portions 42, 44 which are disposed transversely to the secondary longitudinal axis R. In addition, the second end 38 may include a third portion 46 which extends between the first and second portions 42, 44. The third portion 46 may be generally planar and disposed generally parallel to the secondary longitudinal axis R. Preferably, any utilized end ports 24B are secured to the second end 38 at the third portion 46. The first and second portions 42, 44 are disposed to converge towards the third portion 46 so as to provide a funneling effect to any end port 24B located on the third portion 46. In addition, it is preferred that any utilized side port 24A be secured to the first portion 42. With this arrangement, an enlarged space is created below the side port 24A with portions of the second end 38 being recessed. During media introduction, this advantageously minimizes the extent of cell adherence to portions of the body 12 adjacent to the side port 24A. In addition, this space may act as an air reservoir from which air may be distributed amongst the insert layers 16.

Preferably, the top 20 and the bottom 22 are formed generally planar, and more preferably parallel, to permit level stacking of a plurality of the vessels 10. Ribs or other features may be included on one or both of the top 20 and the bottom 22 to facilitate stacking alignment and stability. Also, a pouch or other containment member may be secured or otherwise supported by the top 20 during use with substance being introduced therefrom via tubing through either of both of the side ports 24A and the end ports 24B.

As will be appreciated by those skilled in the art, the various features discussed herein may be used singularly or in various combinations.

What is claimed is:

1. A multi-layered cell culture vessel comprising:
a body enclosing a closed volume, said body including at least one insert layer located within said closed volume to separate portions thereof, and at least one port allowing communication with said closed volume from exteriorly of said body; and,
at least one grip protruding from said body configured to be grippingly engaged by a user in transporting the multi-layered cell culture vessel.

2. A cell culture vessel as in claim 1, wherein said grip is arcuately formed with ends fixed to said body and a space being defined between said grip and body configured to receive a hand of a user.

3. A cell culture vessel as in claim 1, wherein said at least one port includes a side port protruding from said body in a direction generally parallel to said grip, said grip protruding no further from said body than said side port with a cap mounted thereto.

4. A cell culture vessel as in claim 1, wherein said grip is spaced from said at least one port.

5. A cell culture vessel as in claim 1, wherein said body including first and second ends spaced apart along a longitudinal axis.

6. A cell culture vessel as in claim 5, wherein said at least one port includes a side port, said side port being generally tubular and extending along a secondary longitudinal axis which is disposed transversely to said longitudinal axis.

7. A cell culture vessel as in claim 6, wherein said at least one port includes an end port secured to said second end of said body, said end port allowing communication with said closed volume from exteriorly of said body.

8. A cell culture vessel as in claim 7, wherein said second end includes a first planar portion disposed transversely to said secondary longitudinal axis of said side port, said side port being secured to said body at said first portion.

9. A cell culture vessel as in claim 8, wherein said second end further includes a second planar portion disposed transversely to said secondary longitudinal axis of said side port, said second portion being located along said secondary longitudinal axis further from said side port than said first portion.

10. A cell culture vessel as in claim 9, wherein said second end includes a third portion extending between said first and second portions.

11. A cell culture vessel as in claim 10, wherein said third portion is generally planar and disposed generally parallel to said secondary longitudinal axis.

12. A cell culture vessel as in claim 11, wherein said end port is secured to said second end at said third portion.

13. A multi-layered cell culture vessel comprising:
a body enclosing a closed volume, said body including at least one generally planar insert layer located within said volume to separate portions thereof, and at least one side port allowing communication with said closed volume from exteriorly of said body, wherein, said body defining a generally rectangular footprint as viewed in a first direction with a longitudinal dimension which is greater than a transverse dimension, said side port being generally tubular and extending along a longitudinal axis which is disposed generally parallel to the transverse dimension.

14. A multi-layered cell culture vessel comprising:
a body enclosing a closed volume, said body including at least one generally planar insert layer located within said volume to separate portions thereof, said body including first and second ends spaced apart along a longitudinal axis, said insert extending from said first end and being spaced from said second end so as to define a gap therebetween, and said body including at least one side port allowing communication with said closed volume from exteriorly of said body, said side port being generally tubular and extending along a secondary longitudinal axis which is disposed transversely to said longitudinal axis and which is disposed to intersect said gap defined by said insert layer.
